(19) United States
(12) Patent Application Publication
(10) Pub. No.: US 2003/0212051 A1
(43) Pub. Date: Nov. 13, 2003

(54) Bile acid containing prodrugs with enhanced bioavailabilty

(76) Inventors: James E Polli, Elliot City, MD (US); Andrew Coop, Columbia, MD (US); Dean Y Maeda, Bothel, WA (US); Kimberly A Lentz, Durham, CT (US)

Correspondence Address: SUGHRUE MION, PLLC, 2100 PENNSYLVANIA AVENUE, N.W., WASHINGTON, DC, 20037, US

(21) Appl. No.: 10/240,859

(22) PCT Filed: Apr. 6, 2001
(86) PCT No.: PCT/US01/11327

(51) Int. Cl. A61K 31/56; A61K 31/58; C07J 41/00

(52) U.S. Cl. 514/169; 514/176; 540/106

(57) ABSTRACT

Many compounds have poor bioavailability or variable bioavailability because of poor absorption of the compound in the small intestine. Conjugation of the compound with bile acid to form a prodrug will increase the bioavailibility of the compound and/or reduce the bioavailability variability of the compound because of the active transport of the prodrug by the intestinal bile acid transporter and because of increased lipophilic nature of the prodrug. A linker group can be used between the bile acid and the compound. One example of a bile acid containing prodrug is acyclovir valylchenodeoxycholate, where valine is the linker group. Another example of this prodrug is atenolol cholic acid amide.

### BACKGROUND OF THE INVENTION

1. Field of Invention

This invention relates to the method of increasing the bioavailability and reducing the bioavailability variability of compounds by conjugating or linking a bile acid to the compound. This invention also relates to the new composition of matter obtained by attaching a bile acid to another compound to generate a prodrug. This invention further relates to the usage of the bile acid transporter to actively move a prodrug out of the lumen of the small intestine.

2. Description of the Related Art

Insufficient or variable intestinal permeability is a reason for inadequate oral drug bioavailability. Many methods are available to increase bioavailability of various drugs [1]. While some of these methods improve the bioavailability of some drugs, not one method works for all compounds.

Some compounds that exhibit poor bioavailability, sub-optimal permeability, or variable bioavailability may have some or all of the following characteristics [2]:

1. Less than complete oral absorption.

2. Permeability less than a suitable reference marker (e.g. metoprolol tartrate [e.g. a permeability of 40×10⁻⁶ cm/sec across Caco-2 monolayers]).

3. Molecular weight greater than 500 Daltons.

4. Hydrogen donors greater than five.

5. Hydrogen bond acceptors greater than 10.

6. Being substrates for P-glycoprotein efflux, Multi-drug Resistance-associated Protein (MRP) efflux, or other efflux systems.

7. Not being substrates of or ligands for a carrier or transporter.

One potential method to increase the bioavailability of compounds with these characteristics is to conjugate a bile acid to the drug or compound to create a prodrug. A bile acid conjugated prodrug allows for an increase in bioavailability and/or a reduction in the variability of the compound because of active transport of the prodrug by the intestinal bile acid transporter (IBAT). The intestinal absorption of bile acids is a sodium dependent process involving the human intestinal bile acid transporter (hIBAT) and Na⁺/K⁺ ATP-ase [3].

The bile acid transporter is an ideal candidate for drug targeting because the human IBAT has a high transport capacity of 10 grams per day [4, 5], and bile acids are one of the largest molecules taken up by a carrier mediated system [6]. In light of this biology, the bile acid transporter appears to be a promising mechanism to improve oral drug absorption by incorporating a bile acid moiety with an active drug in a prodrug fashion. The bile acid prodrug approach to targeting the bile acid transporter in various tissues has been investigated with peptides [7, 8, 9], HMG-CoA reductase inhibitors [8], and chlorambucil [10]. This work was directed either at extremely small molecules or for targeting delivery of drugs to the liver. Other attempts at drug therapy involve inhibiting the bile acid transporter to reduce cholesterol synthesis in the liver [11].

One compound that may benefit from bile acid conjugation to improve uptake in the small intestine is acyclovir. Acyclovir is an anti-viral compound, used to inhibit herpes virus proliferation. Its target is not the liver but all tissues within the body. Typical treatment requires 200 mg doses administered five times daily, with a bioavailability after oral administration of 20% [12].

A different prodrug strategy has proved successful at improving the oral bioavailability of acyclovir [13, 14]. Valacyclovir, the L-valine ester prodrug of acyclovir, has an oral bioavailability of 54% [15]. This improved bioavailability for valacyclovir allows for a more convenient dosing regimen of 1000 mg, twice daily, with similar clinical efficacy as previously found for the acyclovir parent compound [16]. Valacyclovir is a substrate for the human intestinal peptide transporter (PepT1), with a Kᵢ=4.08 mM in PepT1 expressing Xenopus laevis oocytes [17] and Kᵢ=1.10 mM in stable lines of CHO/PepT1 [18].

In comparison to PepT1, IBAT has the potential advantages of higher capacity and micromolar affinity. Therefore, it is possible to further enhance the oral bioavailability of acyclovir by synthesizing a cholic acid ester prodrug to target the IBAT. It is also possible to increase the bioavailability of other compounds by conjugating the compounds to a bile acid.

Furthermore, animals other than humans have an intestinal bile acid transporter. One can use the animal intestinal bile acid transport and a bile acid conjugated prodrug to increase the adsorption of the prodrug and bioavailability of the compound in animals.

### BRIEF DESCRIPTION OF THE INVENTION

It is an object of this invention to increase the bioavailability of a compound by conjugating a compound to a bile acid to create a prodrug. It is a further object of this invention that the prodrug is administered orally to an animal or human. It is a further object of this invention that the intestinal bile acid transporter binds to the prodrug and moves the prodrug from the lumen of the small intestine into brush border cells. It is a further object of this invention that the prodrug or compound moves from inside the brush border cells into the blood stream.

It is an object of this invention to increase the bioavailability of a compound by conjugating a compound to a bile acid to create a prodrug. It is another object of this invention that a metabolically labile bond exist between the compound and the bile acid. It is a further object of this invention that the prodrug is administered orally to an animal or human. It is a further object of this invention that the intestinal bile acid transporter binds to the prodrug and moves the prodrug from the lumen of the small intestine into brush border cells. It is a further object of this invention that the prodrug or compound moves from inside the brush border cells into the blood stream.

It is an object of this invention to increase the bioavailability of a compound by linking a compound to a bile acid via a linker group to create a prodrug. It is a further object of this invention that the prodrug is administered orally to an animal or human. It is a further object of this invention that the intestinal bile acid transporter binds to the prodrug and moves the prodrug from the lumen of the small intestine into brush border cells. It is a further object of this invention that the prodrug or compound moves from inside the brush border cells into the blood stream. It is another object of this invention that the linker group be any bifunctional chemical moiety that achieves one or more of the following four functions: (1) facilitates the synthesis of the prodrug, (2) aid or enhance the binding of the prodrug to the intestinal bile acid transporter (IBAT), (3) make it easier for the compound to disassociate from the bile acid after the prodrug has passed out of the lumen of the small intestine, (4) enhance the solubility of the prodrug inside the body. It is another object of this invention that the linker group be any size, but more preferably be less than 200 daltons. It is a further object of this invention that a metabolically labile bond exists between the linker group and the compound, between the linker group and the bile acid, or within the linker group itself.

It is an object of this invention to increase the bioavailability of a biologically active compound by conjugating a biologically active compound to a bile acid to create a prodrug. It is a further object of this invention that a metabolically labile bond exist between the biologically active compound with the bile acid. It is a further object of this invention that the prodrug is administered orally to an animal or human. It is a further object of this invention that the intestinal bile acid transporter binds to the prodrug and moves the prodrug from the lumen of the small intestine into brush border cells. It is a further object of this invention that the prodrug or compound moves from inside the brush border cells into the blood stream.

It is an object of this invention to conjugate a compound to a bile acid to form a prodrug.

It is an object of this invention to have a pharmaceutical compound containing a compound and a bile acid.

It is another object of this invention to have a pharmaceutical compound containing a compound, a metabolically labile bond, and a bile acid.

It is an object of this invention to have a pharmaceutical compound containing an agent, a linker group, and a bile acid. It is another object of this invention to have a pharmaceutical compound containing an agent, a linker group, a bile acid, and a metabolically labile bond.

It is an object of this invention to have a pharmaceutical compound containing an agent with biological activity and a bile acid.

It is another object of this invention to have a pharmaceutical compound containing an agent with biological activity, a metabolically labile bond, and a bile acid.

It is an object of this invention to have a pharmaceutical compound containing an agent with biological activity, a linker group, and a bile acid. It is another object of this invention to have a pharmaceutical compound containing an agent with biological activity, a linker group, a bile acid, and a metabolically labile bond.

It is an object of this invention to utilize the intestinal bile acid transporter to actively uptake and remove from the lumen of the small intestine a bile acid containing prodrug.

It is an object of this invention to have a method for increasing the bioavailability of a compound by using the intestinal bile acid transporter to actively uptake and remove from the lumen of the small intestine a bile acid containing prodrug.

It is another object of this invention to use a linker group to link a compound to a bile acid to create a prodrug. It is further object of this invention that the linker group be any bifunctional chemical moiety that achieves one or more of the following four functions: (1) facilitates the synthesis of the prodrug, (2) aid or enhance the binding of the prodrug to the intestinal bile acid transporter (IBAT), (3) make it easier for the compound to disassociate from the bile acid after the prodrug has passed out of the lumen of the small intestine, (4) enhance the solubility of the prodrug inside the body. It is another object of this invention that the linker group be any size, more preferably less than 200 daltons. It is also an object of this invention that the linker group, when attached to the compound or the bile acid, result in a metabolically labile bond being present.

It is an object of this invention to reduce the bioavailability variability of a compound by conjugating a compound to a bile acid to create a prodrug. It is a further object of this invention that the prodrug is administered orally to an animal or human. It is a further object of this invention that the intestinal bile acid transporter binds to the prodrug and moves the prodrug from the lumen of the small intestine into brush border cells. It is a further object of this invention that the prodrug or compound moves from inside the brush border cells into the blood stream.

It is an object of this invention to reduce the bioavailability variability of a compound by conjugating a compound to a bile acid to create a prodrug. It is another object of this invention that a metabolically labile bond exist between the compound and the bile acid. It is a further object of this invention that the prodrug is administered orally to an animal or human. It is a further object of this invention that the intestinal bile acid transporter binds to the prodrug and moves the prodrug from the lumen of the small intestine into brush border cells. It is a further object of this invention that the prodrug or compound moves from inside the brush border cells into the blood stream.

It is an object of this invention to reduce the bioavailability variability of a compound by linking a compound to a bile acid via a linker group to create a prodrug. It is a further object of this invention that the prodrug is administered orally to an animal or human. It is a further object of this invention that the intestinal bile acid transporter binds to the prodrug and moves the prodrug from the lumen of the small intestine into brush border cells. It is a further object of this invention that the prodrug or compound moves from inside the brush border cells into the blood stream. It is another object of this invention that the linker group be a bifunctional chemical moiety. It is another object of this invention that the linker group be any size, but more preferably be less than 200 daltons. It is a further object of this invention that a metabolically labile bond exists between the linker group and the compound, between the linker group and the bile acid, or within the linker group itself.

It is an object of this invention to reduce the bioavailability variability of a biologically active compound by conjugating a biologically active compound to a bile acid to create a prodrug. It is a further object of this invention that a metabolically labile bond exist between the biologically active compound with the bile acid. It is a further object of this invention that the prodrug is administered orally to an animal or human. It is a further object of this invention that the intestinal bile acid transporter binds to the prodrug and moves the prodrug from the lumen of the small intestine into brush border cells. It is a further object of this invention that the prodrug or compound moves from inside the brush border cells into the blood stream.

It is an object of this invention to reduce or prevent an adverse drug-drug interaction by linking or conjugating a bile acid to at least one of the compounds which is involved in the adverse drug-drug interaction to create a prodrug. It is a further object of this invention to use the intestinal bile acid transporter to uptake the prodrug, thereby avoiding a transporter that is involved in the adverse drug-drug interaction.

It is an object of this invention to reduce or prevent an adverse drug-nutrient interaction by linking or conjugating a bile acid to a compound which is involved in the adverse drug-nutrient interaction to create a prodrug. It is a further object of this invention to use the intestinal bile acid transporter to uptake the prodrug, thereby avoiding a transporter that is involved in the adverse drug-nutrient interaction.

It is an object of this invention to increase the lipophilicity of a compound by linking or conjugating a bile acid to the compound. It is a further object of this invention that the increased lipophilicity will increase the bioavailability and reduce the variability of the bioavailability of the compound.

It is an object of this invention that the compound component of the prodrug will be cleaved from the prodrug after the prodrug binds to the intestinal bile acid transporter.

It is an object of this invention that the prodrug be coated with a substance that protects the prodrug from the acidic environment of the stomach and that does not inhibit absorption of the prodrug in the intestine.

It is an object of this invention to have a pharmaceutical compound of acyclovir valyldeoxycholate.

It is an object of this invention to have a pharmaceutical compound of acyclovir valylchenodeoxycholate.

It is an object of this invention to have a pharmaceutical compound of atenolol cholic acid amide.

### DESCRIPTION OF SEVERAL VIEWS OF THE DRAWINGS

FIG. 1A illustrates the general structure of bile acids.

FIG. 1B illustrates the general structure of the prodrug.

FIG. 1C generalizes the synthesis of acyclovir valylchenodexycholate.

FIG. 1D generalizes the synthesis of acyclovir valyldeoxycholate.

FIG. 1E generalizes the synthesis of atenolol cholic acid amide.

FIG. 2 illustrates the competitive inhibition of uptake of ³H-taurocholate by acyclovir valylchenodeoxycholate (acyclovir vCDC) (●), chenodeoxycholate (CDC) (∘), and valacyclovir (Δ).

FIG. 3 illustrates the competitive inhibition of uptake of ³H-taurocholate by acyclovir valyldeoxycholate (acyclovir vDC) (●), deoxycholate (DC) (∘), and valacyclovir (Δ).

FIG. 4 illustrates the competitive inhibition of uptake of ³H-taurocholate by atenolol cholic acid amide (●), cholate (∘), and atenolol (Δ).

FIG. 5 shows the concentration dependence of ³H-taurocholate uptake in COS-hIBAT (COS cells transfected with hIBAT) in HBSS (♦) and in MHBSS (no sodium) (▪).

FIG. 6 illustrates the inhibition constant (Kᵢ) of various bile acids and other agents by inhibiting ³H-taurocholate uptake in COS cells transfected with hIBAT.

### DETAILED DESCRIPTION OF THE INVENTION

In the preferred embodiment, this invention is a prodrug containing a bile acid attached to a compound. In an alternative embodiment, more than one bile acid can be attached to a compound. Alternatively, more than one compound can be attached to one bile acid. Alternatively, a complex of one or more bile acids can be attached to one or more compounds.

When used wherein, the word “compound” includes, but is not limited to, a pharmaceutical drug, a biologically active agent, a metabolic precursor to a pharmaceutical drug, a metabolic precursor to a biologically active agent, or any other agent which one would want to administer to an animal or human. A compound may be therapeutic or diagnostic in nature. A compound can also be nutritionally beneficial to an animal or human.

It is preferable that a metabolically labile bond exist between a compound and the bile acid to which the compound is attached or conjugated. By way of example only, a metabolically labile bond can be an ester, amide, carbamate, carbonate, ether, urea, anhydride, or sulfur containing derivatives, such as thioamides, thioesters, thiocarbamates, and thioureas.

In an alternative embodiment, a linker group exists between a compound and the bile acid to which the compound is attached. The linker group can be any bifunctional chemical moiety but preferably has a molecular weight less than 200 daltons. Four reasons exist for using a linker group; any bifunctional chemical moiety that achieves any one of these reasons is considered a linker group. First, the linker group can facilitates the synthesis of the prodrug. Second, the linker group can aid or enhance the binding of the prodrug to the intestinal bile acid transporter (IBAT). Third, the linker group can make it easier for the compound to disassociate from the bile acid after the prodrug has passed out of the lumen of the small intestine. Fourth, the linker group can enhance the solubility of the prodrug inside the body.

Different types of linker groups can be used. Linker groups can be, for example, natural and unnatural amino acids, di-acids, di-amines, di-alcohols, sulphate, phosphate, sulfur containing moieties, amino alcohols, hydroxyacids, and polymers. Examples of the amino acids are valine, glycine, taurine, alanine, leucine, tyrosine, aspartate, glutamate, lysine, arginine, asparagine, cysteine. Examples of the di-acids are oxalic acid, fumaric acid, succinic acid, maleic acid, and tartaric acid. Examples of the di-amines include ethylenediamine, propylenediamine, 1,3-diaminopropane, 1,4-diaminobutane, 1,5-diaminopentane, piperazine, homopiperazine, and 3-aminopiperidine. Examples of the di-alcohols are ethyleneglycol, propyleneglycol, 1,4-butanediol, polyethyleneglycol, and 1,5-pentanediol. Examples of the sulfur containing moieties are mercaptoacetic acid, mercaptopropanoic acid, mercaptobenzyl alcohol, 2-mercaptoethanol, 3-mercaptopropanol, and 4-mercaptobutanol. Examples of the amino alcohols are 2-aminoethanol, 3-aminopropanol, 4-aminobutanol, 4-hydroxypiperidine, and 3-hydroxypiperidine. Examples of the hydroxyacids include 3-hydroxypropanoic acid, 4-hydroxybutanoic acid, and 5-hydroxypentanoic acid. One example of polymers is polyethylene glycol.

It is preferably that the linker group has a metabolically labile bond for easy cleavage of the prodrug into the compound and the bile acid. By way of example only, a metabolically labile bond can be an ester, amide, carbamate, carbonate, ether, urea, anhydride, or sulfur containing derivatives, such as thioamides, thioesters, thiocarbamates, and thioureas.

These bile acid containing prodrugs are administered orally and absorbed in the small intestine. The intestinal bile acid transporter actively transports the prodrug from inside the lumen of the small intestine into brush border cells. Once inside brush border cells, the prodrug may pass passively through the cell membrane down a concentration gradient into the blood stream or be actively transported out of the brush border cells into the blood stream. This active transport of the prodrug out of the lumen of the small intestine occurs because of the affinity of the intestinal bile acid transporter for its ligand, bile acids.

Intracellular or extracellular enzymes cleave one of the bonds between the bile acid and the compound either (1) after the prodrug is inside the brush border cells, (2) attached to the outside of the brush border cells, (3) located in proximity to the brush border cells, (4) in the blood stream, or (5) at other location inside the body, except cleavage should not occur in the stomach or lumen of the small intestine.

This active transport of a prodrug by the intestinal bile acid transport produces enhanced bioavailability of the compound when compared to the unconjugated compound. It also reduces bioavailability variability and provides for an alternative mechanism for compound permeation, including the avoidance of efflux pumps such as P-glycoprotein used by some drugs (e.g., fexofenadine) and other drug transporters and/or nutrient transporters. By avoiding other drug transporters and/or nutrient transporters, drug-drug and/or drug-nutrient interactions can be reduced or eliminated.

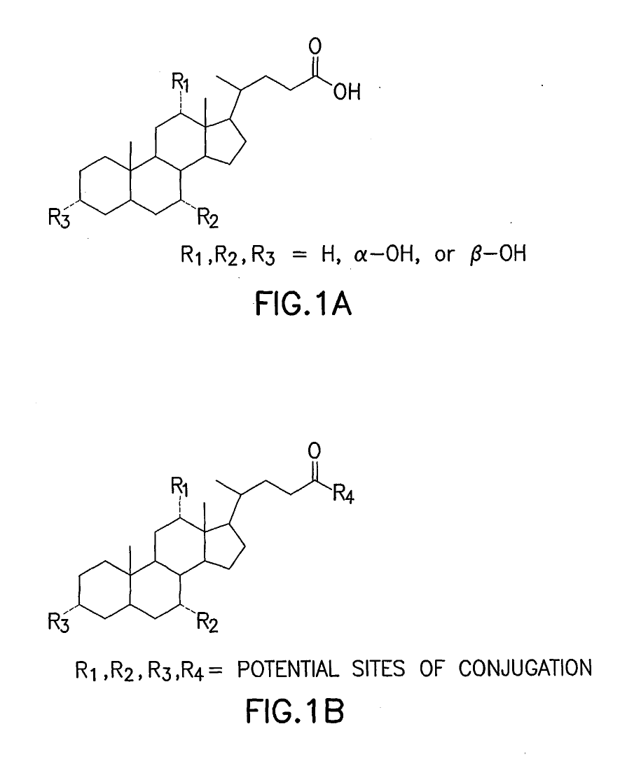

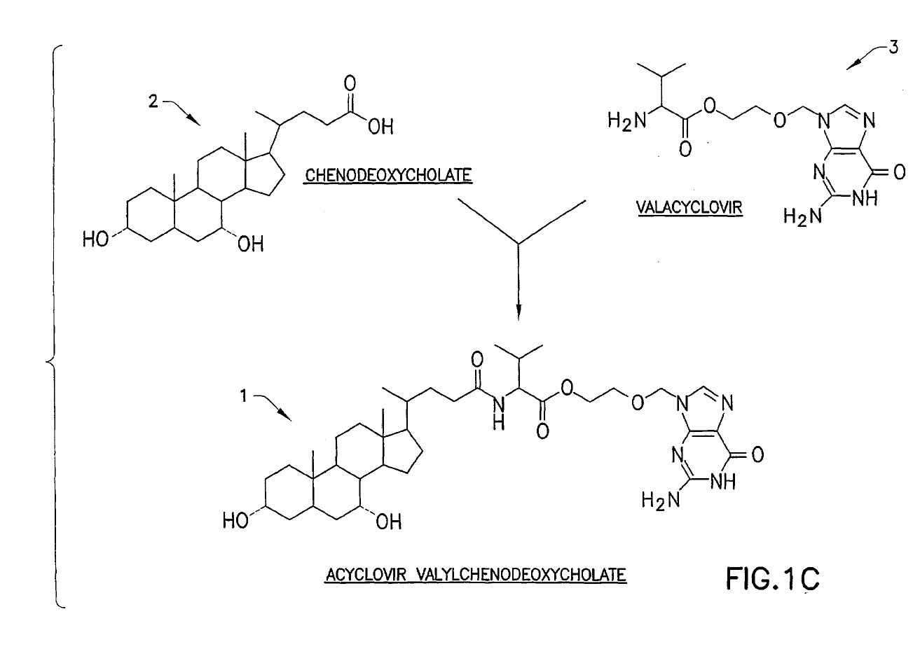

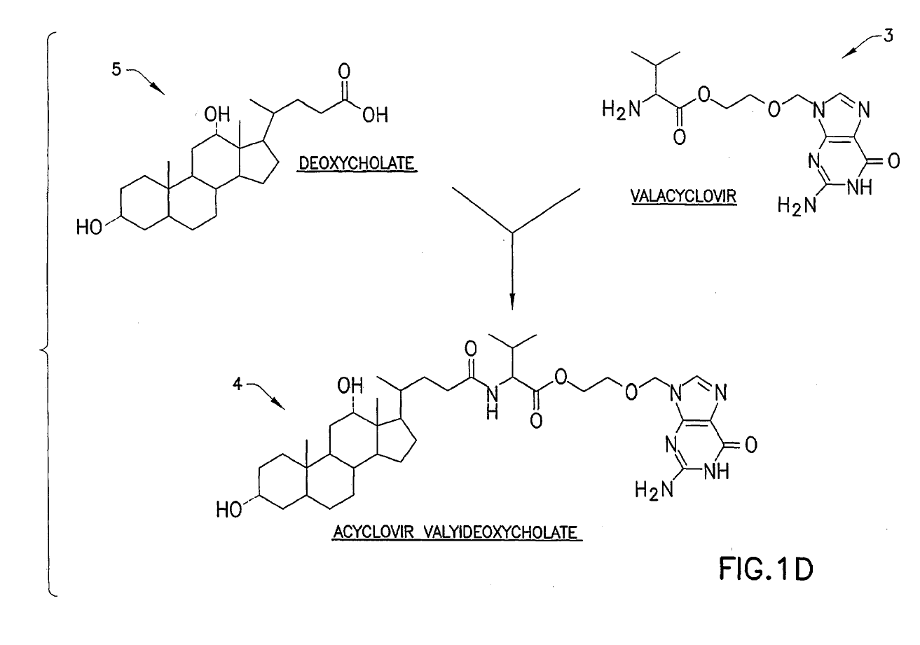

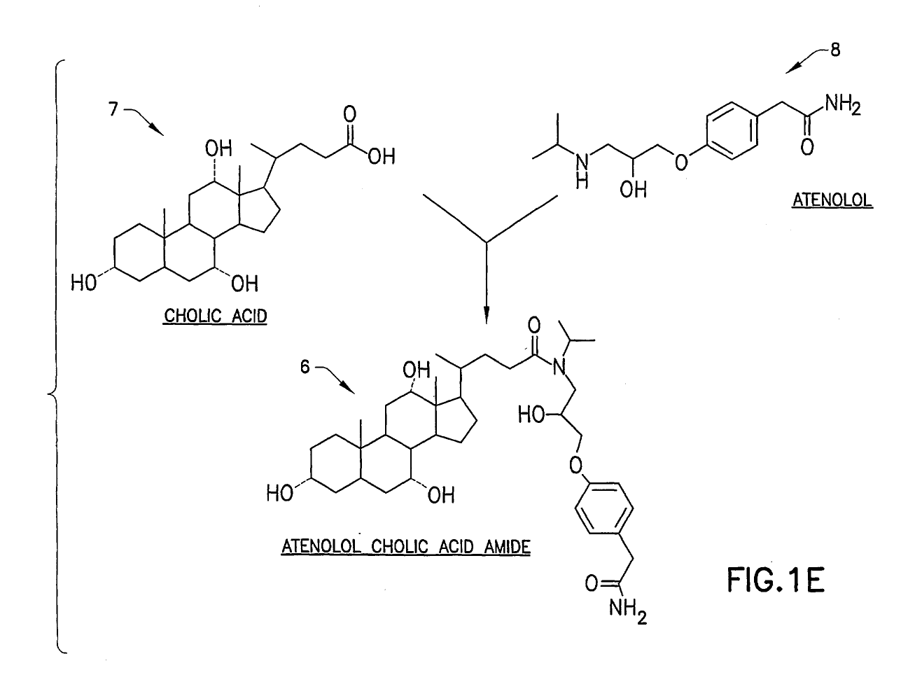

Any bile acid may be conjugated or linked to a compound. More than one bile acid may be conjugated or linked to one compound. Also more than one compound may be conjugated or linked to one bile acid. The bile acids are, by way of example, cholate, glycocholate, taurocholate, deoxycholate, glycodeoxycholate, taurodeoxycholate, chenodeoxycholate, ursodeoxycholate, glycochenodeoxycholate, taurochenodeoxycholate, and lithocholate. The general structures of the bile acids are shown in FIG. 1A.

The following structural requirements generally promote recognition of a bile acid by the intestinal bile acid transporter (IBAT): negatively charged side groups on the bile acid; at least one alpha oriented hydroxyl group at the steroid nucleus at position 3, 7, or 12; and a cis configuration of rings A and B of the steroid nucleus [3].

By way of example, compounds that have improved bioavailability as a result of conjugating or linking the compound to a bile acid are those compounds which contain (Group 1) an alcohol function which can be chemically coupled to the bile acid to give an ester; or (Group 2) a primary or secondary amine function which can be chemically coupled to the bile acid to give an amide; or (Group 3) an acid function which can be chemically coupled to the bile acid to give an anhydride.

Compounds which contain an alcohol function (Group 1) may include, but are not limited to, anti-viral agents (e.g. 6-deoxyacyclovir, ganciclovir, dihydroxybutylguanine, foscarnet, penciclovir, famciclovir, zidovudine, idoxuridine, 5-trifluorothymidine, vidarabine, cytarabine, ribavirin). Compounds which contain a primary or secondary amine function (Group 2) may include, but are not limited to, H-2 antagonists (e.g. derivatives of cimetidine, ranitidine, nizatidine, famotidine, roxatidine). Compounds which contain an acid function (Group 3) may include, but are not limited to, bisphosphonates (e.g. alendronate, etidronate, pamidronate, tiludronate, clonronate) and ACE inhibitors (e.g. enalapril, captopril, lisonopril). This list is not exhaustive and is not meant to limit the scope of this invention, but is provided by way of illustration only.

Any salts, solvates, isomeric compositions, and physical forms of these compounds and/or their derivatives can be conjugated or linked to a bile acid to form a prodrug. One can also use known in the art field enteric coating to protect prodrugs from the acidity of the stomach.

FIG. 1A illustrates the general structure of bile acids. Bile acids can vary in their substituents at R₁, R₂, and R₃ (i.e. positions 12, 7, and 3, respectively). For example, for chenodeoxycholate, R₁ is H, R₂ is α-OH, and R₃ is α-OH. For deoxycholate, R₁ is α-OH, R₂ is H, and R₃ is α-OH. For cholate, R₁, R₂, and R₃ are all α-OH. Many natural and synthetic bile acids exist and are well-known in the art field. Any of these natural or synthetic bile acids can be used to create a prodrug. However, it may be preferable to have at least one alpha orientated hydroxyl group in the bile acid and this alpha orientated hydroxyl group can be located at R₁, R₂, or R₃.

FIG. 1B illustrates the general structure of bile acid component of the prodrug. Either directly or via a linker group, a compound can be attached to any natural or synthetic bile acid at either R₁, R₂, R₃, or R₄. While it may be preferable to have only one compound conjugated or linked to one bile acid, more than one compound can be conjugated or linked to the same bile acid. In addition, while it may be preferable to have the compound linked or conjugated to a bile acid at R₄ (because of potential structural requirements necessary for recognition of a bile acid by the intestinal bile acid transporter), it is possible to link or conjugate compounds to a bile acid at R₁, R₂, or R₃. When a compound is linked or conjugated to either R₁, R₂, or R₃, then it is preferable that R₄ be any chemical moiety which can aid or enhance the binding of the prodrug to the IBAT and/or can increase the solubility of the prodrug inside the body. Examples of such chemical moieties include hydroxyl, any amino acid (such as glycyl, valyl, alanyl, tauryl, and leucyl), any di-amino acid (such as glycyl-valyl, glycyl-glycyl, valyl-glycyl, alanyl-glycyl, alanyl-valyl, and leucyl-valyl), any tri-amino acid (such as glycyl-glycyl-glycyl, glycyl-valyl-glycyl, glycyl-glycyl-alanyl, valyl-glycyl-leucyl, alanyl-glycyl-glycyl, alanyl-valyl-glycyl, and leucyl-valyl-valyl). It is preferable that this chemical moiety have a molecular weight less than 1500 daltons.

FIGS. 1C, 1D, and 1E generalize the synthesis of three prodrugs, acyclovir valylchenodeoxycholate, acyclovir valyldeoxycholate, and atenolol cholic acid amide, which have increased bioavailability and reduced bioavailability variability as a result of conjugating or linking the compounds, acyclovir and atenolol, to a bile acid. For these three prodrugs, the compounds are attached to a bile acid (chenodeoxycholate, deoxycholate, or cholic acid) at R₄. For acyclovir valylchenodeoxycholate and acyclovir valyldeoxycholate, valine is utilized as a linker group. Furthermore, both acyclovir valylchenodeoxycholate and acyclovir valyldeoxycholate use an ester as a metabolically labile bond that can be cleaved to release acyclovir. For atenolol cholic acid amide, no linker group is used, but rather atenolol is conjugated directly to cholic acid. Furthermore, atenolol cholic acid uses an amide as a metabolically labile bond that can be cleaved to release atenolol.

### Acyclovir Valylchenodeoxycholate Synthesis

As in FIG. 1C, to synthesize acyclovir valylchenodeoxycholate, 1, isobutylchloroformate (iBuOCOCl; 130 μL, 1 mmol) is added dropwise to a cooled (−15° C.) solution of chenodeoxycholate, 2, (1 mmol) and triethylamine (140 μL, 1 mmol) in N,N-dimethylformamide (DMF) (10 mL) under an nitrogen (N₂) atmosphere. After 1.5 minutes, valacyclovir, 3, (0.42 g, 1.3 mmol) and triethylamine (NEt₃; 280 μL, 2 mmol) are then added to the reaction mixture as a solution in DMF (5 mL). The reaction is kept at −15 ° C. for 0.5 hours, then is warmed to room temperature for 1 hour. The triethylammonium chloride formed during the reaction is filtered off, and the filtrate is concentrated by rotary evaporation. The crude material is then purified using silica gel flash chromatograpy with MeOH/CHCl₃ (1:4, 250 mL) as the eluent.

The acyclovir valylchenodeoxycholate, 1, prodrug synthesis is monitored using thin layer chromatography (TLC) plates coated with silica gel GHLF-0.25 mm plates (60 F₂₅₄) manufactured by Analtech, Inc. (Newark, Del.). Fast-atom bombardment mass spectrometry (FAB-MS) and high resolution mass spectrometry (HRMS) spectra are obtained on a Jeol SX 102 mass spectrometer in the positive ion mode. Proton nuclear magnetic resonance (NMR) spectrometry is performed in d₆-dimethyl sulfoxide (DMSO) on a 300 MHz General Electric Aquerius model spectrometer controlled by a Macintosh Power Mac 7100 using MacNMR v. 5.0 software. The purity of the bile acid conjugate is determined by analysis on a Beckman System Gold high pressure liquid chromatography (HPLC) system consisting of a model 126 solvent module, model 168 detector, and model 507 autosampler. The HPLC column used is a Vydac analytical column (C₁₈, 300 Å, 5 μm, 4.6×250 mn) equipped with a guard cartridge. Solvent A is aqueous 0.1% trifluoroacetic acid (TFA) and solvent B is acetonitrile containing 0.1% TFA. The conjugate is eluted using a linear gradient of 5 to 75% B over 50 minutes at a flow rate of 1.0 mL/min and detected at 214 mm.

The amount of acyclovir valylchenodeoxycholate, 1, purified is 0.62 g (89%). Additionally, TLC R_{f}(MeOH/CHCl₃, 1:4)=0.46; HPLC Rₜ=32.1 min (99.3% purity); and ESI-MS [M+H]=700.4. HRMS (calculated for C₃₇H₅₉O₇N₆): 699.4445. found 699.4454. The NMR spectra contained peaks consistent with both chenodeoxychloate and valacyclovir portions. Coupling through the amino acid amine (and not the aniline) was confirmed through the presence of the NH₂ signal at 5.3 ppm.

### Acyclovir Valyldeoxycholate Synthesis

As in FIG. 1D, to synthesize acyclovir valyldeoxycholate, 4, isobutylchloroformate (iBuOCOCl; 130 μL, 1 mmol) is added dropwise to a cooled (−15° C.) solution of deoxycholate, 5, (1 mmol) and triethylamine (140 μL, 1 mmol) in N,N-dimethylformamide (DMF) (10 mL) under an nitrogen (N₂) atmosphere. After 1.5 minutes, valacyclovir, 3, (0.42 g, 1.3 mmol) and triethylamine (NEt₃; 280 μL, 2 mmol) are then added to the reaction mixture as a solution in DMF (5 mL). The reaction is kept at −15° C. for 0.5 hours, then is warmed to room temperature for 1 hour. The triethylammonium chloride formed during the reaction is filtered off, and the filtrate is concentrated by rotary evaporation. The crude material is then purified using silica gel flash chromatograpy with MeOH/CHCl₃ (1:4, 250 mL) as the eluent.

The acyclovir valyldeoxycholate, 4, prodrug synthesis is monitored using thin layer chromatography (TLC) plates coated with silica gel GHLF-0.25 mm plates (60 F₂₅₄) manufactured by Analtech, Inc. (Newark, Del.). Fast-atom bombardment mass spectrometry (FAB-MS) and high resolution mass spectrometry (HRMS) spectra are obtained on a Jeol SX 102 mass spectrometer in the positive ion mode. Proton nuclear magnetic resonance (NMR) spectrometry is performed in d₆-dimethyl sulfoxide (DMSO) on a 300 MHz General Electric Aquerius model spectrometer controlled by a Macintosh Power Mac 7100 using MacNMR v. 5.0 software. The purity of the bile acid conjugate is determined by analysis on a Beckman System Gold high pressure liquid chromatography (HPLC) system consisting of a model 126 solvent module, model 168 detector, and model 507 autosampler. The HPLC column used is a Vydac analytical column (C₁₈, 300 Å, 5 μm, 4.6×250 mm) equipped with a guard cartridge. Solvent A is aqueous 0.1% trifluoroacetic acid (TFA) and solvent B is acetonitrile containing 0.1% TFA. The conjugate is eluted using a linear gradient of 5 to 75% B over 50 minutes at a flow rate of 1.0 mL/min and detected at 214 nm.

The amount of acyclovir valyldeoxycholate, 4, purified is 0.59 g (85%). Additionally, TLC R_{f}MeOH/CHCl₃, 1:4)=0.47; HPLC Rₜ=33.0 min (98.1% purity); FAB-MS [M+H]⁺=699.54; HRMS (calculated for C₃₇H₅₉O₆N₇): 699.4445, found: 699.4448. The NMR spectrum contained peaks consistent with both deoxycholic acid and valacyclovir portions. Coupling through the amino acid amine (and not the aniline) was confirmed through the presence of the NH₂ signal at 5.3 ppm.

### Atenolol Cholic Acid Amide Synthesis

As in FIG. 1E, to synthesize atenolol cholic acid amide, 6, isobutylchloroformate (iBuOCOCl; 130 μL, 1 mmol) is added dropwise to a cooled (−15 ° C.) solution of cholic acid, 7, (0.5 mmol) and triethylamine (140 μL, 1 mmol) in N,N-dimethylformamide (DMF) (10 mL) under an nitrogen (N₂) atmosphere. After 1.5 minutes, atenolol, 8, (0.42 g, 1.3 mmol) and triethylamine (NEt₃; 280 μL, 2 mmol) are then added to the reaction mixture as a solution in DMF (5 mL). The reaction is kept at −15° C. for 0.5 hours, then is warmed to room temperature for 1 hour. The triethylammonium chloride formed during the reaction is filtered off, and the filtrate is concentrated by rotary evaporation. The crude material is then purified using silica gel flash chromatograpy with MeOH/CHCl₃ (1:4, 250 mL) as the eluent.

The atenolol cholic acid amide, 6, prodrug synthesis is monitored using thin layer chromotography (TLC) plates coated with silica gel GHLF-0.25 mm plates (60 F₂₅₄) manufactured by Analtech, Inc. (Newark, Del.). Fast-atom bombardment mass spectrometry (FAB-MS) and high resolution mass spectrometry (HRMS) spectra are obtained on a Jeol SX 102 mass spectrometer in the positive ion mode. Proton nuclear magnetic resonance (NMR) spectrometry is performed in d₆-dimethyl sulfoxide (DMSO) on a 300 MHz General Electric Aquerius model spectrometer controlled by a Macintosh Power Mac 7100 using MacNMR v. 5.0 software. The purity of the bile acid conjugate is determined by analysis on a Beckman System Gold high pressure liquid chromatography (HPLC) system consisting of a model 126 solvent module, model 168 detector, and model 507 autosampler. The HPLC column used is a Vydac analytical column (C₁₈, 300 Å, 5 μm, 4.6×250 mm) equipped with a guard cartridge. Solvent A is aqueous 0.1% trifluoroacetic acid (TFA) and solvent B is acetonitrile containing 0.1% TFA. The conjugate is eluted using a linear gradient of 5 to 75% B over 50 minutes at a flow rate of 1.0 mL/min and detected at 214 nm.

The amount of atenolol cholic acid amide, 6, purified is 0.19 g (58%). Additionally, TLC R_{f}AcOH/MeOH/CHCl₃, 1:1:4)=0.47, HPLC Rₜ=34.9 min (98.3% purity); FAB-MS [M+H]⁺=657.6; HRMS (calculated for C₃₈H₆₁N₂O₇): 657.4479, found: 657.4470; IR (CHCl₃) 3394, 2932, 2870, 1671, and 1610 cm⁻¹.

Atenolol, 8, contains both a secondary alcohol and a secondary amine, and coupling to cholic acid, 7, through both of these nucleophiles could occur to give an ester or an amide, respectively. Only the amide is formed during the above reaction of cholic acid and atenolol which is confirmed through the presence of characteristic amide C═O stretching bands at 1671 cm⁻¹ and 1610 cm⁻¹ in the IR spectrum. Also, NMR spectrum is consistent with that expected for a conjugate between cholic acid and atenolol (i.e., the absence of the secondary amine proton signal at 4.92 ppm in the NMR). Coupling through the amine of atenolol (and not the alcohol) is also confirmed by a negative chloranil test, a test for secondary and primary amines. Atenolol is used as the positive control for the chloranil test.

### Bioavailability Assays

To test the increase of bioavailability of acyclovir valylchenodeoxycholate, 1, acyclovir valyldeoxycholate, 4, and atenolol cholic acid amide, 6, the human intestinal bile acid transporter (hIBAT) cDNA (specifically, pCMV5-hIBAT expression plasmid) is transformed into competent DH5α cells according to the Life Technologies protocol (Grand Island, N.Y.), using LipofectAMINE 2000 transfection reagent (Life Technologies, Grand Island, N.Y.). Cell dilutions are streaked on nutrient agar plates containing 50 μg/ml ampicillin, and incubated at 37° C. overnight. Isolated colonies are selected aseptically, and are used to inoculate a 200 ml nutrient broth culture containing 50 μg/ml ampicillin. The culture is incubated for 24 hours at 225 RPM's and 37° C. cDNA is isolated from the broth cultures using a Quigan maxi-prep kit (DNA plasmid maxi kit #12162; Valencia, Calif.). After an isopropanol precipitation and ethanol wash, the DNA is reconstituted in 600 μl of sterile deionized water. DNA concentration is 1.18 μg/μl, determined by spectrophotometry at 260 nm. The 260/280 absorbance ratio is 1.50, indicating the DNA is free from RNA contamination. pCMV5-hIBAT is also digested with the restriction endonucleases, BAMH1 and NDE, and results in bands of appropriate size (4238 bp and 1689 bp) when electrophoresed on a 1% agarose gel.

COS-7 cells are grown in T-75 flasks at 37° C., 5% CO₂ and 95% RH using Dulbecco's Modified Eagle's Medium (DMEM) supplemented with 10% FBS. Cells are passaged at 80-90% confluency using a 0.25% trypsin/0.20% EDTA solution, and are plated at a density of 8×10⁴ cells per well (1.88 cm²). Cells are transfected at 24 hours post seeding. For each well transfected, 0.8 μg hIBAT cDNA is combined with LipofectAMINE 2000 reagent and incubated at room temperature for 20 minutes to allow complexes to form. A volume of 100 μl of hIBAT-lipid complex is added to each well of cells. Transfected cells are incubated at 37° C., 5% CO₂ and 95% RH for 24 hours, until ready for uptake assay.

Uptake studies are performed on COS-hIBAT cells at 24 hours post-transfection. Uptake buffer consists of either a Hank's Balanced Salts Solution (HBSS) containing 137 mM NaCl or a Modified Hank's Balanced Salts Solution (MHBSS) that replaced the sodium chloride with 137 mM tetraethylammonium chloride [19]. Because bile acid transport is sodium dependent, this MHBSS approach allows for the simple modification of the uptake buffer to exclude all sources of sodium, and thus enables for a control uptake assay under sodium-free conditions.

For the Kₘ and Vₘₐₓ studies of taurocholate in the COS-hIBAT cells, cell culture medium is removed from each well and is replaced with 0.5 ml of uptake solution. 0.5 μM ³H-taurocholate is investigated in HBSS. Cells are incubated at 37° C. and 100 RPM's for 10 minutes. Uptake solution is removed, and the cells are washed three times with ice cold HBSS. Cells are lysed, neutralized, and counted for associated radioactivity. Each well is analyzed for protein content using the Lowry method [20]. This uptake assay is performed in triplicate, under both sodium and sodium-free conditions.

The saturable uptake of ³H-taurocholate is determined using the following equation (Formula 1):

$$\frac{ⅆ M}{ⅆ t} = \frac{{V}_{max} \cdot S}{{K}_{m} + S} + {k}_{p} S \qquad ( Formula 1 )$$

where Vₘₐₓ and Kₘ represent the Michaelis-Menten constants, kₚ is the passive uptake rate constant, S is the concentration of taurocholate, and dM/dt is the uptake rate of taurocholate into the COS-hIBAT cells. This approach includes the contribution of passive uptake to the saturable kinetics of taurocholate uptake into the cells.

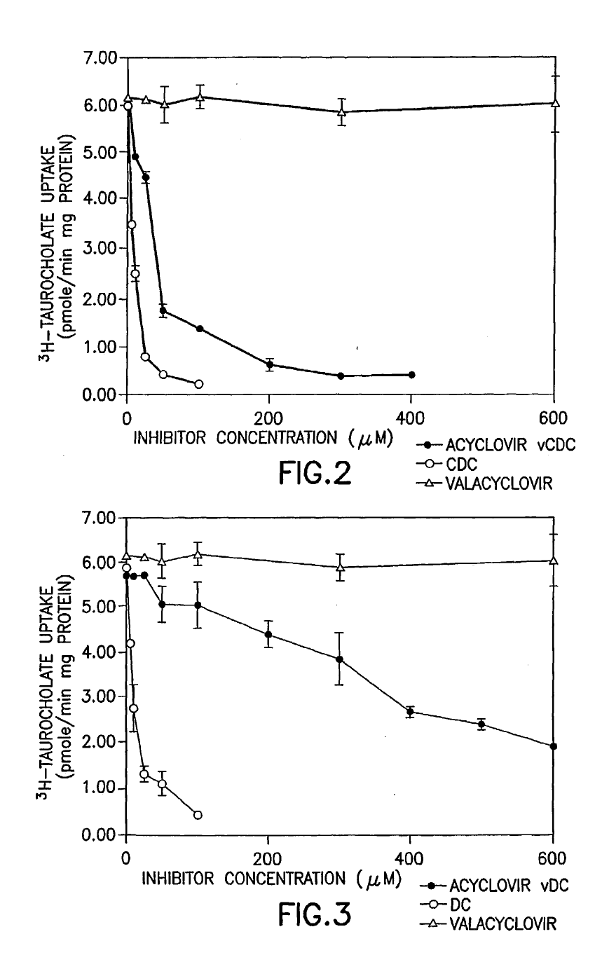

In the competitive inhibition studies (see FIGS. 2, 3, and 4), the concentration of acyclovir valylchenodeoxycholate, 1, varies between 10 μM and 400 μM; the concentration of acyclovir valyldeoxycholate, 4, varies between 10 μM and 600 μM; and the concentration of atenolol cholic acid amide, 6, varies between 10 μM and 400 μM. Chenodeoxycholate, 2, deoxycholate, 5, and cholate inhibition studies are conduced as positive controls. Valacyclovir and atenolol inhibition studies are performed as negative controls.

FIG. 2 illustrates the inhibition study of valacyclovir, 3, (Δ), chenodeoxycholate (CDC), 2, (∘), and acyclovir valylchenodeoxycholate (acyclovir vCDC), 1, (●). There is no inhibition of the bile acid transporter in COS-hIBAT cells after application of up to 600 μM valacyclovir, indicating that valacyclovir is not a substrate for hIBAT. A chenodeoxycholate inhibition study is performed as a positive control for this experiment, and results in Kᵢ=5.4 (±0.4) μM, which indicates that the COS-hIBAT model is suitable for the inhibition study. The calculated Kᵢ for acyclovir valylchenodeoxycholate is Kᵢ=35.6 (±4.4) μM, indicating a very strong interaction of acyclovir valylchenodeoxycholate for the hIBAT transporter.

Similiarly for acyclovir valyldeoxycholate (FIG. 3), inhibition study of valacyclovir, 3, ({), deoxycholate (DC), 5, (∘), and acyclovir valyldeoxycholate (acyclovir vDC), 4, (●) are performed. Acyclovir valyldeoxycholate strongly interacts with hIBAT with a Kᵢ=401 (±50) μM.

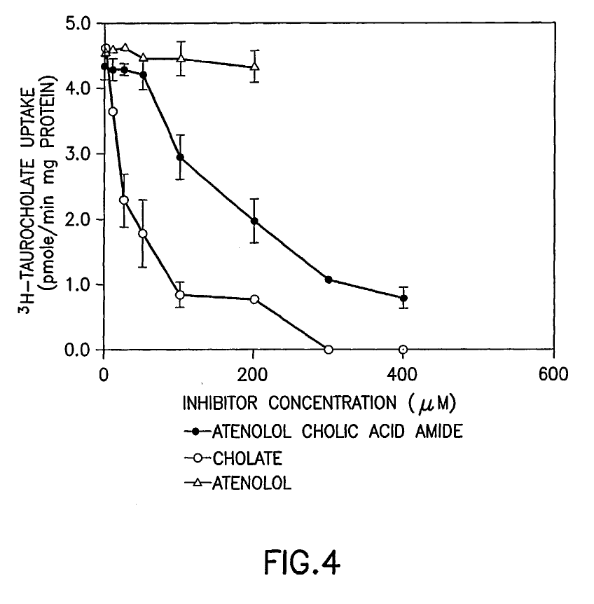

Likewise, in FIG. 4, atenolol cholic acid amide, 6, (●) strongly interacts with hIBAT with a Kᵢ=160 (121) μM, while atenolol, 8, (Δ) itself does not interact. The natural bile acid cholate, 7, (∘) also strongly interacts with hIBAT [Kᵢ=32.1 (±2.4) μM].

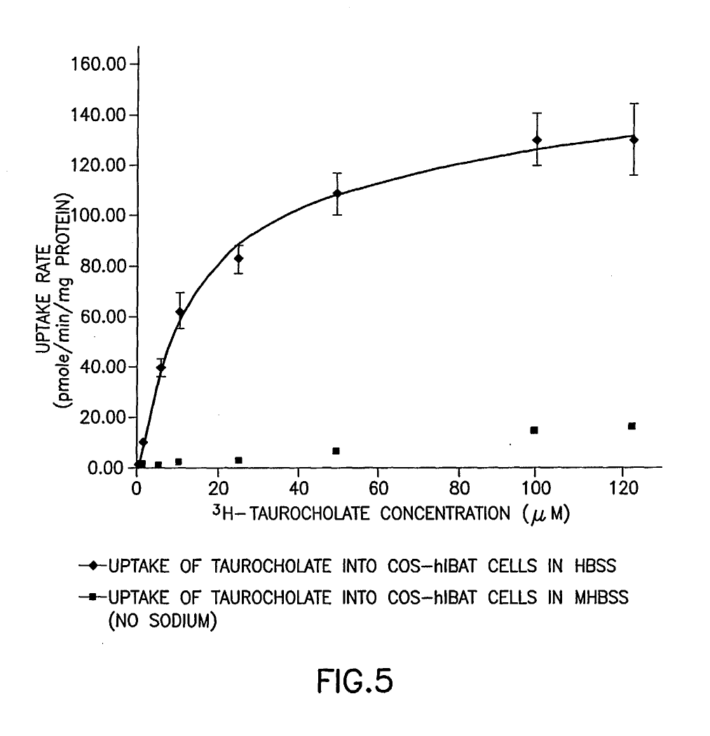

FIG. 5 shows both the sodium dependence and saturation of ³H-taurocholate uptake. The uptake rate is measured at ³H-taurocholate concentrations from 0.1 to 125 μM in HBSS with 137 mM NaCl and in MHBSS which contains no sodium. As expected, carrier mediated ³H-taurocholate uptake is not present in the absence of sodium ions, thus establishing a baseline permeability due to the passive permeability of taurocholate into the cells. A passive uptake rate constant (kₚ) of 0.12 (pmoles/min mg protein)/μM is estimated using linear regression. Control uptake studies are also performed in untransfected COS-7 cells, and in COS-7 cells transfected with the antibiotic resistant vector, pcDNA3. The passive uptake of ³H-taurocholate in these experiments is the same as that obtained under sodium-free conditions (data not shown). The kinetic parameters for carrier mediated uptake are estimated using WinNonlin (version 1.0), and yields a Kₘ=12.0(±2.2) μM and a Vₘₐₓ=126.0(±5.9) pmoles/min/mg protein. These Michaelis-Menten parameters are in close agreement to those obtained previously [21].

Competitive inhibition studies are performed in HBSS containing 0.25 μM ³H-taurocholate, and varying the concentration of unlabeled bile acid from 1 to 100 μM. Glycine, taurine, acyclovir, and atenolol inhibition studies are performed as negative controls. Incubation conditions and analysis are performed as described for the Kₘ and Vₘₐₓ studies. The following equation (Formula 2) is used to estimate the Kᵢ for a series of naturally occurring bile acids:

$$\frac{ⅆ M}{ⅆ t} = \frac{{V}_{max} \cdot ( S / {K}_{m} )}{1 + \frac{S}{{K}_{m}} + \frac{I}{{K}_{i}}} \qquad ( Formula 2 )$$

where Vₘₐₓ and Kₘ are the Michaelis-Menten parameters for taurocholate uptake, S is 0.25 μM ³H-taurocholate, dN/dt is the uptake rate of taurocholate, and I is the concentration of inhibitor applied to the cells.

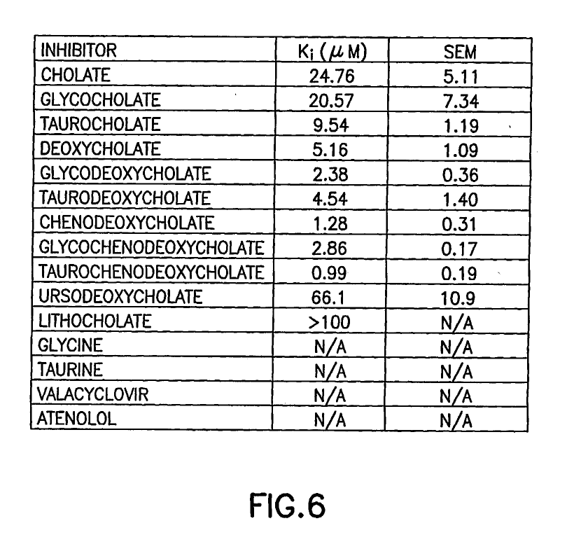

The effect of various bile acids (cholate, glycocholate, taurocholate, deoxycholate, taurodeoxycholate, glycodeoxycholate, chenodeoxycholate, glycochenodeoxycholate, taurochenodeoxycholate, ursodeoxycholate, and lithocholate) on the uptake of ³H-taurocholate into COS-hIBAT cells is determined (see FIG. 6). Inhibition studies using glycine, taurine, valacyclovir, and atenolol are also performed as negative controls. FIG. 6 shows the Kᵢ value (±SEM) for each compound tested. All bile acids inhibit the uptake of ³H-taurocholate into COS-hIBAT cells. However, the uptake of ³H-taurocholate is not reduced in the presence of glycine or taurine. Uptake of ³H-taurocholate decreases in the presence of lithocholate, but never reaches 50% maximal velocity at a lithocholate concentration of 100 μM. Lithocholate concentrations above 100 μM were not investigated. All bile acids inhibit the uptake of ³H-taurocholate in COS-hIBAT cells. Valacyclovir and atenolol do not inhibit the uptake of ³H-taurocholate. In these inhibition studies, ³H-taurocholate concentration is held constant at 0.25 μM and inhibitor concentration is varied between 1 and 100 μM; except for glycine and taurine, which range in concentration from 50 to 200 μM, valacyclovir which range from 10 to 600 μM, and atenolol which range from 10 to 200 μM. Inhibition studies are performed in triplicate. N/A denotes not applicable, because there was no evidence of inhibition.

Compounds can be conjugated or linked via a linker group to bile acids to improve the bioavailability and reduce the bioavailability variability of the compounds. It is preferable that a metabolically labile bond exist between the bile acid and the compound (with or without a linker group) for easy cleavage of the compound from the bile acid. While it is preferable that the compound or compound and linker group be attached to the R₄ of the bile acid, one can also attach the compound or compound and linker group at R₁, R₂, or R₃. Also, one can link or conjugate different or the same compounds to multiple positions on the bile acid. Furthermore, one may attach more than one bile acid to a compound.

The prodrugs can be coated with various coating compounds known in the art field to protect the prodrug from the acidic environment in the stomach. These coating compounds dissolve in the basic environment in the small intestine, thereby permitting the prodrug to be available for uptake by the IBAT.

The prodrugs can also be converted into a pharmaceutically acceptable salt or pharmaceutically acceptable solvate or other physical forms (e.g. polymorphs by way of example only and not limitation) via known in the art field methods. Pharmaceutically acceptable carriers can be used along with the prodrugs. In making the compositions of the present invention, the prodrug can be mixed with an excipient, diluted by an excipient or enclosed within such a carrier which can be in the form of a capsule, sachet, paper or other container. When the excipient serves as a diluent, it can be a solid, semi-solid, or liquid material, which acts as a vehicle, carrier, or medium for the prodrug. Thus, the compositions can be in the form of tablets, pills, powers, lozenges, sachets, cachets, elixirs, suspensions, emulsions, solutions, syrups, soft and hard gelatin capsules, and other orally ingestible formulations.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include lubricating agents such as talc, magnesium stearate, and mineral oil; wetting agents; emulsifying and suspending agents; preserving agents such as methyl- and propyl-hydroxybenzoates, sweetening agents; and flavoring agents. The compositions of the present invention can also be formulated so as to provide quick, sustained or delayed release of the prodrug after administration to the patient by employing procedures known in the art.

The compositions are preferably formulated in a unit dosage form, each dosage containing from about 0.005 mg to 100 mg, more usually about 1.0 mg to about 300 mg, of the prodrug. Unit dosage form generally refers to physically discrete units suitable as unitary dosages for human and animal patients, each unit containing a predetermined quantity of the prodrug calculated to produce the desired therapeutic effect, in association with a suitable pharmaceutical excipient.

A prodrug is used for treatment of human or animal patients which are in need of treatment with the compound that is contained in the prodrug. The specific purpose of the treatment, and the dose range to be administered, depends on the identity of the compound and the condition for which the patient is to be treated.

While a list of compounds for which this prodrug approach will work is too large to be contained here, below is a short list of compounds and their “drug” class:

foscarnet, valacyclovir, acyclovir, ganciclovir, penciclovir, famciclovir (anti-virals); alendronate, etidronate disodium, pamidronate, risedronate, tiludronate, clodronic acid (bisphosphonates); cimetidine, ranitidine (H-2 antagonists); enalaprilate, captopril, lisonopril (ACE inhibitors); losartan, E-3171 (angiotensin II antagonists); levofloxacin, norfloxacin (quinalone antibiotic which have decreased absorption with antacids); formycin B; acetbutalol, pindolol, alprenolol, atenolol, nadolol (beta adrenergic blockers); bretylium tosylate (antiarrhythmic agents); cefuroxime sodium (cephalosporins); chlorothiazide, hydrochlorothiazide, furosemide (diuretic agents); gabapentin, lamotrigine (anticonvulsant); didanosine (nucleoside reverse transcriptase inhibitors); neviriapine (non-nucleoside reverse transcriptase inhibitors); ritinavir, saquinavir, amprinavir (HIV protease inhibitors); tacrolimus, cyclosporin (immunosuppresants); zafirlukast (leukotriene receptor antagonists); leuprorelin actetate (LHRH analogues); dDAVP (1-deamino-8-D-arginine-vasopressin; desmopressin), calcitonin, thyrotropin releasing hormone (polypeptide hormones); loratidine, cetirizine (non-sedating antihistamines); penicillin V, amoxicillin, cefacor, cefixime, cefuroxime axetil, cefuroxime sodium, ampicillin (antibiotics); terbutaline hemisulfate (adrenergic agonist agents); metformin (anti-diabetics); celecoxib, refecoxib (COX-2 inhibitors); sumatriptan, naratriptan, araztriptan, zolmitriptan (anti-migraines); 6-mercaptopurine; ziprasidone; RGD mimetic (alpha IIb beta 3-antagonists); leuenkephalin analogues; alpha-methyldopa; 5-fluorouracil (fluoropoyrimidines); tacrine (acetylcholinesterase inhibitors); DZ-2640 (the ester-type oral carbapenem prodrug of an active parent compound, DU-6681, and other carbapenems); vitamin B 12 (nutrients and minerals); 7-chlorokynurenic acid; oseltamivir or its active moiety; RGD (Arg-Gly-Asp) analogs (glycoprotein (GP) IIb/IIIa agonists and antagonists; platelet aggregation inhibitors); sibrafiban (oral platelet aggregation inhibitors); nelarabine, 9-beta-D-arabinofuranosyl guanine (ara-G), and ara-G; mycophenolate mofetil (MMF) and its active immunosuppressant mycophenolic acid (MPA); nabumetone and its active metabolite 6-methoxy-2-naphthylacetic acid (anti-osteoarthritis agents); adefovir (9-[2-phosphonylmethoxyethyl]-adenine [PMEA]) and adefovir dipivoxil [bis-(POM)-PMEA], and cidofovir (antiviral nucleotides); cromoglicate lisetil and cromoglycic acid (anti-arthritis agents); oseltamivir or its active moiety; its parent Ro 64-0802 (inhibitors of influenza virus neuraminidase); peptidomimetics; nucleic acids;

This is for illustrative purposes only and is not meant to be exhaustive. Other compounds will have increased bioavailability and/or reduced bioavailability variability with bile acid conjugation.

All references cited herein are incorporated by reference in their entirety.

References

[1] Remington: The Science and Practice of Pharmacy (A. R. Gennaro, editor). Mack Publishing Compant, Easton, Pa. 1995.

[2] Lipinski, C. A.; Lombardo, F.; Dominy, B. W.; Feeney, P. J. Adv. Drug Del. Rev. 1997, 23, 3-25.

[3] L. Lack, Properties and biological significance of the ileal bile salt transport system, Environ Health Perspect 33 (1979) 79-90.

[4] W. Kramer, G. Wess, A. Enhsen, E. Falk, A. Hoffmann, G. Neckermann, G. Schubert and M. Urmann, Modified bile acids as carriers for peptides and drugs, J Cont Rel 46 (1997) 17-30.

[5] S. Matern and W. Gerok, Pathophysiology of the enterohepatic circulation of bile acids, Rev Physiol Biochem Pharmacol 85 (1979) 126-204.

[6] G. Burckhardt, W. Kramer, G. Kurz and F. A. Wilson, Inhibition of bile salt transport in brush border membrane vesicles from rat small intestine by photoaffinity labeling, J Biol Chem 258 (1983) 3618-3622.

[7] M. Kagedahl, P. W. Swaan, C. T. Redemann, M. Tang, C. S. Craik, F. C. Szoka, Jr. and S. Oie, Use of the intestinal bile acid transporter for the uptake of cholic acid conjugates with HIV-1 protease inhibitory activity, Pharm Res 14 (1997) 176-80.

[8] W. Kramer, G. Wess, A. Enhsen, K. Bock, E. Falk, A. Hoffmann, G. Neckermann, D. Gantz, S. Schulz and L. Nickau, Bile Acid derived HMG-CoA reductase inhibitors., Biochim Biophys Acta 1227 (1994) 137-154.

[9] D. C. Kim, A. W. Harrison, M. J. Ruwart, K. F. Wilkinson, J. F. Fisher, I. J. Hidalgo and R. T. Borchardt, Evaluation of the bile acid transporter in enhancing intestinal permeability to renin-inhibitory peptides., J Drug Target 1 (1993) 347-359.

[10] W. Kramer, G. Wess, G. Schubert, M. Bickel, F. Girbig, U. Gutjahr, S. Kowalewski, K. H. Baringhaus, A. Enhsen and H. G. H, Liver-specific drug targeting by coupling to bile acids., J Biol Chem 267 (1992) 18598-18604.

[11] M. C. Lewis, L. E. Brieaddy and C. Root, Effects of 2164U90 on ileal bile acid absorption and serum cholesterol in rats and mice, J Lipid Res 36 (1995) 1098-1105.

[12] P. deMiranda and M. R. Blum, Pharmacokinetics of acyclovir after intravenous and oral administration, J Antimicrob Chemother 12 (1983) 29-37.

[13] M. A. Jacobson, Valaciclovir (BW256U87): the L-valyl ester of acyclovir., J Med Virol Supplement 1 (1993) 150-153.

[14] H. Gao, A. K. Mitra. Synthesis of acyclovir, gancyclovir, and their prodrugs: A review. Synthesis (2000): 329-359.

[15] J. Soul-Lawton, E. Seaber, N. On, R. Wootton, P. Rolan and J. Posner, Absolute bioavailability and metabolic disposition of valaciclovir, the L-valyl ester of acyclovir, following oral administration to humans, Antimicrob Agents Chemother 39 (1995) 2759-2764.

[16] K. H. Fife, R. A. Barbarash, T. Rudolph, B. Degregorio and R. Roth, Valaciclovir versus acyclovir in the treatment of first-episode genital herpes infection. Results of an international, multicenter, double-blind, randomized clinical trial, Sex Transm Dis 24 (1997) 481-486.

[17] P. V. Balimane, I. Tamai, A. Guo, T. Nakanishi, H. Kitada, F. H. Leibach, A. Tsuji and P. J. Sinko, Direct evidence for peptide transporter (PepT1)-mediated uptake of a nonpeptide prodrug, valacyclovir, Biochem Biophys Res Comm 250 (1998) 246-251.

[18] H. Han, R. L. deVrueh, J. K. Rhie, K. M. Covitz, P. L. Smith, C. P. Lee, D. M. Oh, W. Sadee and G. L. Amidon, 5′-Amino acid esters of antiviral nucleosides, acyclovir, and AZT are absorbed by the intestina; PEPT1 peptide transporter, Pharm Res 15 (1998) 1154-1159.

[19] C. E. Chandler, L. M. Zaccaro and J. B. Moberly, Transepithelial transport of cholyltaurine by Caco-2 cell monolayers is sodium dependent, Am J Physiol 264 (1993) G1118-25.

[20] O. H. Lowry, N. J. Rosebrough, A. L. Farr and R. J. Randall, Protein measurement with the folin phenol reagent, J Biol Chem 193 (1951) 265-275.

[21] M. H. Wong, P. Oelkers and P. A. Dawson, Identification of a mutation in the ileal sodium-dependent bile acid transporter gene that abolishes transport activity, J Biol Chem 270 (1995) 27228-27234.

[22] F. Gaspari and M. Bonati, Correlation between n-octanol/water partition coefficient and liquid chromatographic retention for caffeine and its metabolites, and some structure-pharmacokinetic considerations, J Pharm Pharmacol 39 (1987) 252-60.

[23] K. A. Lentz, J. Hayashi, L. J. Lucisano and J. E. Polli, Development of a more rapid, reduced serum culture system for Caco-2 monolayers and application to the Biopharmaceutics Classification System, Int J Pharm 200 (2000) 41-51.

[24] G. A. Sawada, C. L. Barsuhn, B. S. Lutzke, M. E. Hooughton, G. E. Padbury, N. F. H Ho and T. J. Raub, Increased lipophilicity and subsequent cell partitioning decrease passive transcellular diffusion of novel, highly lipophilic antioxidants, J Pharm Exp Ther 288 (1999) 1317-1326.

[25] G. A. Sawada, L. R. Williams, B. S. Lutzke and T. J. Raub, Novel, highly lipophilic antioxidants readily diffuse across the blood-brain barrier and access intracellular sites, J Pharm Exp Ther 288 (1999) 1327-1333.

While the invention has been described in detail, and with reference to specific embodiments thereof, it will be apparent to one of ordinary skill in the art that various changes and modifications can be made therein without departing from the spirit and scope thereof. The artisan will further acknowledge that the Examples recited herein are demonstrative only and are not meant to be limiting.

We, the inventors, claim

1. A method of increasing the bioavailability of a compound, said method comprising:
conjugating a bile acid to said compound to form a prodrug, and
orally administering said prodrug to an animal or human.

2. The method of claim 1 further comprising the step of:
coating said prodrug with a coating agent prior to orally administering said prodrug.

3. A method of reducing the bioavailability variability of a compound, said method comprising:
conjugating a bile acid to said compound to form a prodrug, and
orally administering said prodrug to an animal or human.

4. The method of claim 3 further comprising the step of:
coating said prodrug with a coating agent prior to orally administering said plug.

5. A method of increasing the bioavailability of a compound, said method comprising:
linking a bile acid to a compound to form a prodrug, and
orally administering said prodrug to an animal or human.

6. The method of claim 5 further comprising the step of:
coating said prodrug with a coating agent prior to orally administering said prodrug.

7. A method of reducing the bioavailability variability of a compound, said method comprising:
linking a bile acid to a compound to form a prodrug, and
orally administering said prodrug to an animal or human.

8. The method of claim 7 further comprising the step of:
coating said prodrug with a coating agent prior to orally administering said prodrug.

9. A method of eliminating adverse interactions between two compounds wherein said adverse interactions result from intestinal absorption of at least one of said compounds comprising:
linking a bile acid to at least one of said compounds to form a prodrug, and
orally administering said prodrug to an animal or human.

10. A method of eliminating adverse interactions between a compound and a nutrient wherein said adverse interactions result from intestinal absorption of said compound comprising:
linking a bile acid to said compound to form a prodrug, and
orally administering said prodrug to an animal or human.

11. A method of eliminating adverse interactions between two compounds wherein said adverse interactions result from intestinal absorption of at least one of said compounds comprising:
conjugating a bile acid to at least one of said compounds to form a prodrug, and
orally administering said prodrug to an animal or human.

12. A method of eliminating adverse interactions between a compound and a nutrient wherein said adverse interactions result from intestinal absorption of said compound comprising:
conjugating a bile acid to said compound to form a prodrug, and
orally administering said prodrug to an animal or human.

13. The method of claims 9, 10, 11, and 12, further comprising:
coating said prodrug with a coating agent prior to orally administering said prodrug.

14. A pharmaceutical compound comprising:
acyclovir valyldeoxycholate.

15. A pharmaceutical compound comprising:
acyclovir valylchendeoxycholate.

16. A pharmaceutical compound comprising:
atenolol cholic acid amide.

17. A pharmaceutical compound comprising:
a compound;
a linker group; and
a bile acid.

18. The pharmaceutical compound of claim 17 further comprising:
wherein said pharmaceutical compound contains a metabolically labile bond.

19. The pharmaceutical compound of claim 18 further comprising:
wherein said metabolically labile bond is selected from a group comprising an amide, ester, carbamate, carbonate, ether, thio, urea, anhydride, thioamide, thioester, thiocarbamate, and thiourea.

20. The pharmaceutical compound of claim 17 further comprising:
wherein said linker group has a molecular weight of less than 200 daltons.

21. A pharmaceutical compound comprising:
a compound; and
a bile acid, wherein said bile acid is chemically bound to said compound through a metabolically labile bond.

23. The pharmaceutical compound of claim 21 further comprising:
wherein said metabolically labile bond is selected from a group comprising an amide, ester, carbamate, carbonate ether, thio, urea, anhydride, thioamide, thioester, thiocarbamate, and thiourea.

24. The pharmaceutical compound of claims 17, 18, 19, 20, 21, and 23 further comprising:
wherein said bile acid is selected from the group comprising cholate, glycocholate, taurocholate, deoxycholate, glycodeoxycholate, taurodeoxycholate, chenodeoxycholate, glycochenodeoxycholate, taurochenodeoxycholate, ursodeoxycholate and lithocholate.

25. A method of increasing the bioavailability of a compound, said method comprising:
attaching a linker group to said compound;
attaching a bile acid to said liner group to form a prodrug, and
orally administering said prodrug to an animal or human.

26. The method of claim 25 further comprising:
wherein said linker group has a molecular weight of less than 200 daltons.

27. The method of claim 25 further comprising:
wherein ad prodrug contains a metabolically labile bond.

28. The method of claim 27 further comprising:
wherein said metabolically labile bond is selected from a group comprising an amide, ester, carbamate, carbonate, ether, thio, urea, anhydride, thioamide, thioester, thiocarbamate, and thiourea.

29. A method of reducing the bioavailability variability of a compound, said method comprising:
attaching a linker group to said compound;
attaching a bile acid to said linker group to form a prodrug, and
orally administering said prodrug to an animal or human.

30. The method of claim 29 further comprising:
wherein said linker group has a molecular weight of less than 200 daltons.

31. The method of claim 29 further comprising:
wherein said prodrug contains a metabolically labile bond.

32. The method of claim 31 further comprising:
wherein said metabolically labile bond is selected from a group comprising an amide, ester, carbamate, carbonate, ether, thio, urea, anhydride, thioamide, thioester, thiocarbamate, and
thiourea.

33. A method of increasing the bioavailability of a compound, said method comprising:
attaching a linker group to a bile acid;
attaching said compound to said linker group to form a prodrug; and
orally administering said prodrug to an animal or human.

34. The method of claim 33 further comprising:
wherein said linker group has a molecular weight of less than 200 daltons.

35. The method of claim 33 further comprising:
wherein said prodrug contains a metabolically labile bond.

36. The method of claim 35 further comprising:
wherein said metabolically labile bond is selected from a group comprising an amide, ester, carbamate, carbonate, ether, thio, urea, anhydride, thioamide, thioester, thiocarbamate, and thiourea.

37. A method of reducing the bioavailability variability of a compound, said method comprising:
attaching a linker group to a bile acid;
attaching said compound to said linker group to form a prodrug; and
orally administering said prodrug to an animal or human.

38. The method of claim 37 further comprising:
wherein said linker group has a molecular weight of less than 200 daltons.

39. The method of claim 37 further comprising:
wherein said prodrug contains a metabolically labile bond.

40. The method of claim 39 further comprising:
wherein said metabolically labile bond is selected from a group comprising an amide, ester, carbamate, carbonate, ether, thio, urea, anhydride, thioamide, thioester, thiocarbamate, and
thiourea.

41. The method of claims 25, 26, 27, 28, 29, 30, 31, 32, 33, 34, 35, 36, 37, 38 39, and 40 further comprising the step of:
coating said prodrug with a coating agent.

42. The method of claims 25, 26, 27, 28, 29, 30, 31, 32, 33, 34, 35, 36, 37, 38 39, 40, and 41 further comprising:
wherein said bile acid is selected from the group comprising cholate, glycocholate, taurocholate, deoxycholate, glycodeoxycholate, taurodeoxycholate, chenodeoxycholate, glycochenodeoxycholate, taurochenodeoxycholate, ursodeoxycholate, and lithocholate.

43. A method for increasing be bioavailability of a compound comprising:
administering a prodrug containing said compound and a bile acid to an animal or human; and
using the intestinal bile acid transporter to actively uptake and remove from the lumen of an intestine said prodrug.

44. A method for reducing the bioavailability variability of a compound comprising:
administering a prodrug containing said compound and a bile acid to an animal or human; and
using the intestinal bile acid transporter to actively uptake and remove from the lumen of an intestine said prodrug.

45. A compound of the formula: 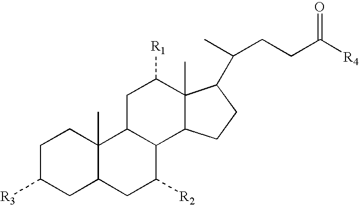
wherein R₁, R₂, and R₃ are independently selected from the group consisting of hydrogen, alpha-hydroxyl, and beta-hydroxyl;
R₄ is selected from the group consisting of an agent having biological activity and a metabolic precursor of an agent having biological activity;
or a pharmaceutically acceptable salt, solvent, or polymorph thereof.

46. A compound of the formula: 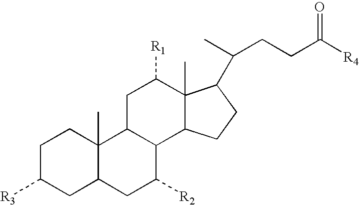
wherein R₂ and R₃ are independently selected from the group consisting of hydrogen, alpha-hydroxyl, and beta-hydroxyl;
R₁ is selected from the group an agent having biological activity and a metabolic precursor of an agent having biological activity;
R₄ is any chemical moiety that enhances binding of the compound to the intestinal bile acid transporter;
or a pharmaceutically acceptable salt, solvent, or polymorph thereof

47. A compound of the formula: 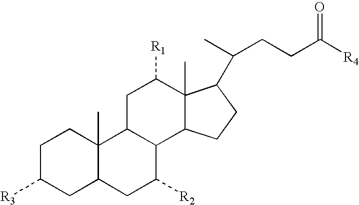
wherein R₁ and R₃ are independently selected from the group consisting of hydrogen, alpha-hydroxyl, and beta-hydroxyl;
R₂ is selected from the group an agent having biological activity and a metabolic precursor of an agent having biological activity;
R₄ is any chemical moiety that enhances binding of the compound to the intestinal bile acid transporter;
or a pharmaceutically acceptable salt, solvent, or polymorph thereof.

48. A compound of the formula: 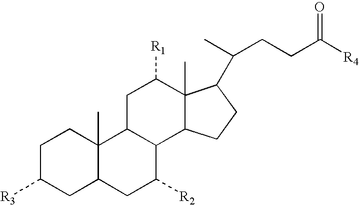
wherein R₁ and R₂ are independently selected from the group consisting of hydrogen, alpha-hydroxyl, and beta-hydroxyl;
R₃ is selected from the group an agent having biological activity and a metabolic precursor of an agent having biological activity;
R₄ is any chemical moiety that enhances binding of the compound to the intestinal bile acid transporter;
or a pharmaceutically acceptable salt, solvent, or polymorph thereof.

49. A compound of the formula: 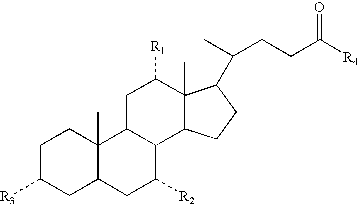
wherein R₂ and R₃ are independently selected from the group consisting of hydrogen, alpha-hydroxyl, and beta-hydroxyl;
R₁ is selected from the group an agent having biological activity and a metabolic precursor of an agent having biological activity;
R₄ is any chemical moiety that increases solubility of the compound;
or a pharmaceutically acceptable salt, solvent, or polymorph thereof.

50. A compound of the formula: 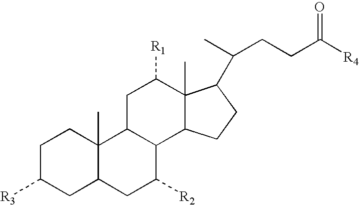
wherein R₁ and R₃ are independently selected from the group consisting of hydrogen, alpha-hydroxyl, and beta-hydroxyl;
R₂ is selected from the group an agent having biological activity and a metabolic precursor of an agent having biological activity;
R₄ is any chemical moiety that increases solubility of the compound;
or a pharmaceutically acceptable salt, solvent, or polymorph thereof.

51. A compound of the formula: 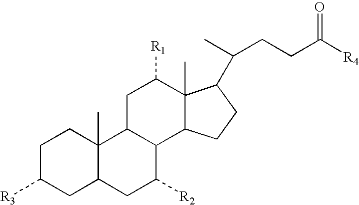
wherein R₁ and R₂ are independently selected from the group consisting of hydrogen, alpha-hydroxyl, and beta-hydroxyl;
R₃ is selected from the group an agent having biological activity and a metabolic precursor of an agent having biological activity;
R₄ is any chemical moiety that increases solubility of the compound:
or a pharmaceutically acceptable salt, solvent, or polymorph thereof.

52. A compound of the formula: 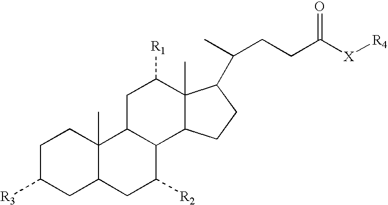
wherein R₁, R₂, and R₃ are independently selected from the group consisting of hydrogen, alpha-hydroxyl, and beta-hydroxyl;
X is any chemical moiety resulting in a metabolically labile bond; and
R₄ is selected from the group consisting of an agent having biological activity or a metabolic precursor of an agent having biological activity;
or a pharmaceutically acceptable salt, solvent, or polymorph thereof.

53. The compound of claim 52 wherein said metabolically labile bond is selected from the group consisting of amide, ester, carbamate, carbonate, ether, thio, urea, anhydride, thioamide, thioester, thiocarbamate, and thiourea.

54. A compound of the formula: 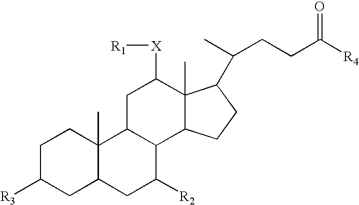
wherein R₂ and R₃ are independently selected from the group consisting of hydrogen, alpha-hydroxyl, and beta-hydroxyl,
R₄ is any chemical moiety that enhances binding of the compound to the intestinal bile acid transporter;
X is any chemical moiety resulting in a metabolically labile bond; and
R₁ is selected from the group consisting of an agent having biological activity or a metabolic precursor of an agent having biological activity;
or a pharmaceutically acceptable salt, solvent, or polymorph thereof.

55. A compound of the formula: 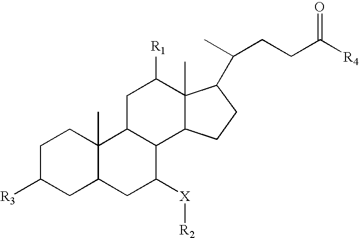
wherein R₁ and R₃ are independently selected from the group consisting of hydrogen, alpha-hydroxyl, and beta-hydroxyl;
R₄ is any chemical moiety that enhances binding of the compound to the intestinal bile acid transporter;
X is any chemical moiety resulting in a metabolically labile bond; and
R₂ is selected tom the group consisting of an agent having biological activity or a metabolic precursor of an agent having biological activity;
or a pharmaceutically acceptable salt, solvent, or polymorph thereof.

56. A compound of the formula: 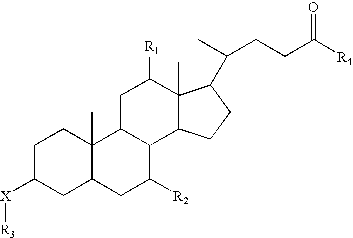
wherein R₁ and R₂ are independently selected from the group consisting of hydrogen, alpha-hydroxyl, and beta-hydroxyl;
R₄ is any chemical moiety that enhances binding of the compound to the intestinal bile acid transporter;
X is any chemical moiety resulting in a metabolically labile bond; and
R₃ is selected from the group consisting of an agent having biological activity or a metabolic precursor of an agent having biological activity;
or a pharmaceutically acceptable salt, solvent, or polymorph thereof.

57. A compound of the formula: 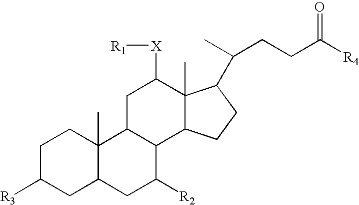
wherein R₂ and R₃ are independently selected from the group consisting of hydrogen, alpha-hydroxyl, and beta-hydroxyl;
R₄ is any chemical moiety that increases solubility of the compound;
X is any chemical moiety resulting in a metabolically labile bond; and
R₁ is selected from the group consisting of all agent having biological activity or a metabolic precursor of an agent having biological activity;
or a pharmaceutically acceptable salt, solvent, or polymorph thereof.

58. A compound of the formula: 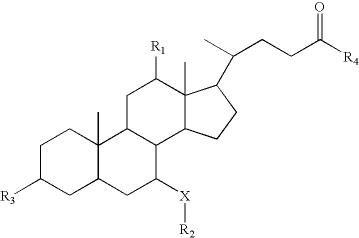
wherein R₁ and R₃ are independently selected from the group consisting of hydrogen, alpha-hydroxyl, and beta-hydroxyl;
R₄ is any chemical moiety that increases solubility of the compound;
X is any chemical moiety resulting in a metabolically labile bond; and
R₂ is selected from the group consisting of an agent having biological activity or a metabolic precursor of an agent having biological activity;
or a pharmaceutically acceptable salt, solvent, or polymorph thereof.

59. A compound of the formula; 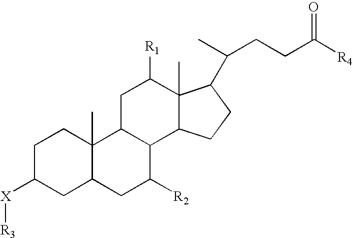
wherein R₁ and R₂ are independently selected from the group consisting of hydrogen, alpha-hydroxyl, and beta-hydroxyl;
R₄ is any chemical moiety that increases solubility of the compound;
X is any chemical moiety resulting in a metabolically labile bond; and
R₃ is selected from the group consisting of an agent having biological activity or a metabolic precursor of an agent having biological activity;
or a pharmaceutically acceptable salt, solvent, or polymorph thereof.

60. The compound of claim 54, 55, 56, 57, 58 and 59 wherein said metabolically labile bond is selected from the group consisting of amide, ester, carbamate, carbonate, ether, thio, urea, anhydride, thioamide, thioester, thiocarbamate, and thiourea.
